(19) United States
(12) Patent Application Publication
(10) Pub. No.: US 2008/0009681 A1
(43) Pub. Date: Jan. 10, 2008

(54) Real time arrhythmia detector for mobile applications

(76) Inventor: Karim Al Hussiny, Houston, TX (US)

Correspondence Address: ARNOLD & KNOBLOCH, L.L.P., 2401 FOUNTAIN VIEW DRIVE, SUITE 630, HOUSTON, TX, 77057, US

(21) Appl. No.: 11/244,320

(22) Filed: Oct. 5, 2005

Related U.S. Application Data
(60) Provisional application No. 60/616,198, filed on Oct. 6, 2004.

(51) Int. Cl. A61B 5/00

(52) U.S. Cl. 600/300

(57) ABSTRACT

Ambulatory patient monitoring apparatus including a portable housing including at least one physiological data input device operative to gather physiological data of the patient, location determination circuitry operative to determine geographic location information of the patient, cellular telephone communications circuitry for communicating the physiological data and the geographic location information to a central health monitoring station, voice communications circuitry whereby the patient conducts voice communications with a clinician at the central health monitoring station, digital signal processing circuitry for processing signals associated with any of the physiological data input device, the location determination circuitry, the cellular telephone communications circuitry, and the voice communications circuitry, and control circuitry for controlling any of the digital signal processing circuitry, the physiological data input device, the location determination circuitry, the cellular telephone communications circuitry, and the voice communications circuitry.

### REFERENCE TO PRIOR RELATED PROVISIONAL APPLICATION

This application claims the benefit of the filing date of co-pending provisional Application No. 60/616,198, filed Oct. 6, 2004.

### BACKGROUND OF THE INVENTION

### Field and Background of the Invention

The present invention relates to the field of real-time interactive monitoring of a patient's physiological condition. More particularly, the invention is directed to apparatus and methods for monitoring a mobile patient's physiological condition and wireless reporting of same to a remote monitoring station. Still more particularly, the invention relates to a pocket size portable Real time cardiac monitoring supervisor device with all 12 leads RT detection-reporting capabilities which is no larger or bulkier than a typical pager, and can be affixed to a patient in a non-obtrusive, manner as he or she goes about their daily routines.

A central and distinguishing feature of the device of the present invention is in its use of extremely efficient algorithms for detection and transmission over GSM or CDMA networks using digital voice channels of silent episodes as they occur. A second distinguishing feature of the device of the present invention is that the receiver at the monitoring center can interrogate the patient on unit diary and real time 12 leads ECG.

Continuously monitoring a patient's physiological condition generally requires the patient's hospitalization, usually at great cost, especially where long term monitoring is required. A wide variety of out-patient monitoring devices have been proposed to monitor the physiology of patients who are physically outside of the hospital. Some out-patient monitoring devices require monitored patients to remain close to a receiving station and thus severely restrict the patient's mobility. Other devices are adapted for monitoring mobile or ambulatory patients while they move about in a vehicle or on foot and have a wide range of operation, but they are generally large and bulky.

One such group of devices includes the so-called halter devices which generally detect and record selected physiological data, such as the patient's ECG, during a predetermined period of time, storing it for examination at later time. Other devices include event recorders. These devices provide for the capture of a patient's physiological data during a significant physiological “event,” such as a cardiac arrhythmia or an episode of patient discomfort. These devices may be patient-activated or activated automatically when physiological data are detected which meet predefined event criteria.

Devices that are non invasive and that in addition enhance the diagnostic perspective continue to dominate demand in the medical devices markets and industries. This demand is more evident for cardiac monitoring devices that can provide detection or early warnings for cardiac emergencies, as these emergencies continue to command the highest premium amongst most medical emergencies.

The industry is presently challenged with mainly technological constraints stemming from inefficient power management of existing portable devices. In addition, real time detection and transmission algorithms are necessary for the realization of increased efficiency in energy utilization. Patient ease of usage, acquisition, detection, and transmission are central design issues that also continue to challenge the industry. At a first glance, algorithms that require minimum computing power and allow for the transmission of data anywhere and anytime are of paramount importance.

Be it sample, time, or frequency based algorithm and while there have been numerous algorithm proposed for such demands, there remains serious drawbacks in that most of these algorithms are power inefficient. Power efficient algorithms that avoid classical brute force computing without sacrificing the diagnostic yield are hence the subject of this disclosure.

For all these reasons there exists a need for a device that is
compact and pocket size (self contained)
provides Real time 12 leads over the GSM cellular network.
is capable of USA FDA approval
is free from 60 Hz AC interference
One push button to initiate indefinite monitoring session(s)
Complete privacy, multiple layers (inherently encrypted) and supersedes US HIPAA compliance www.hipaa.org.
Capable of digital encryption (synchronization)-for transmission directly over a carrier wireless network.
compatible with ECG Storage, Fax, Print, Email, Replay on Server side.

### SUMMARY OF FIGURES

The present invention will be understood and appreciated more fully from the following detailed description taken in conjunction with the appended drawings in which:

FIG. 1 is a simplified pictorial illustration of a personal ambulatory cellular health monitor, constructed and operative in accordance with a preferred embodiment of the present invention;

FIG. 2 is a simplified block diagram illustration of the basic functions of the personal ambulatory cellular health monitor shown in of FIG. 1, constructed and operative in accordance with the present invention;

FIG. 3 is a simplified block diagram illustrating end-to-end communications between a portable monitor and a central medical monitoring station, constructed and operative in accordance with the present invention;

FIG. 4 is a simplified flowchart of the basic algorithm for conditioning waveforms prior to decisions space according to the present invention.

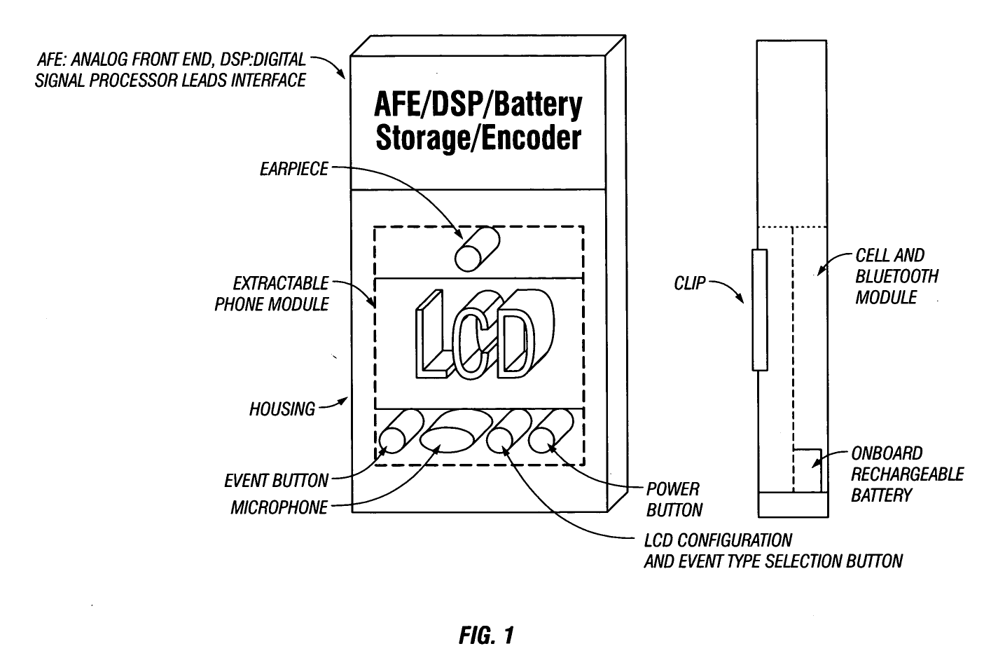

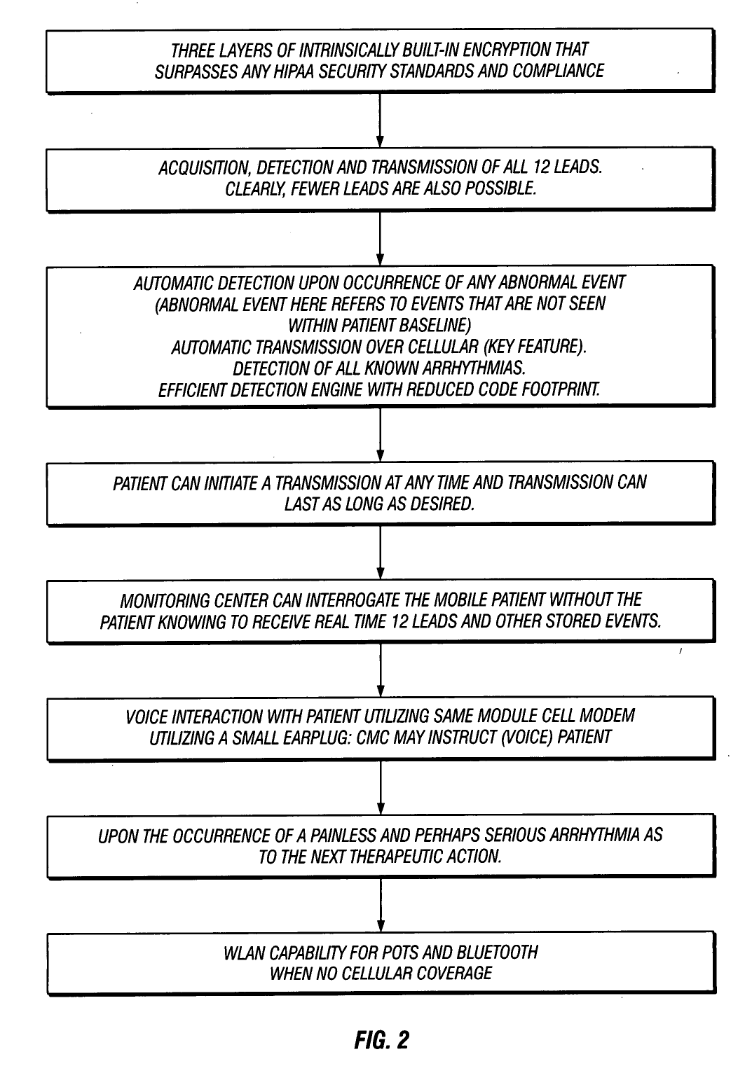

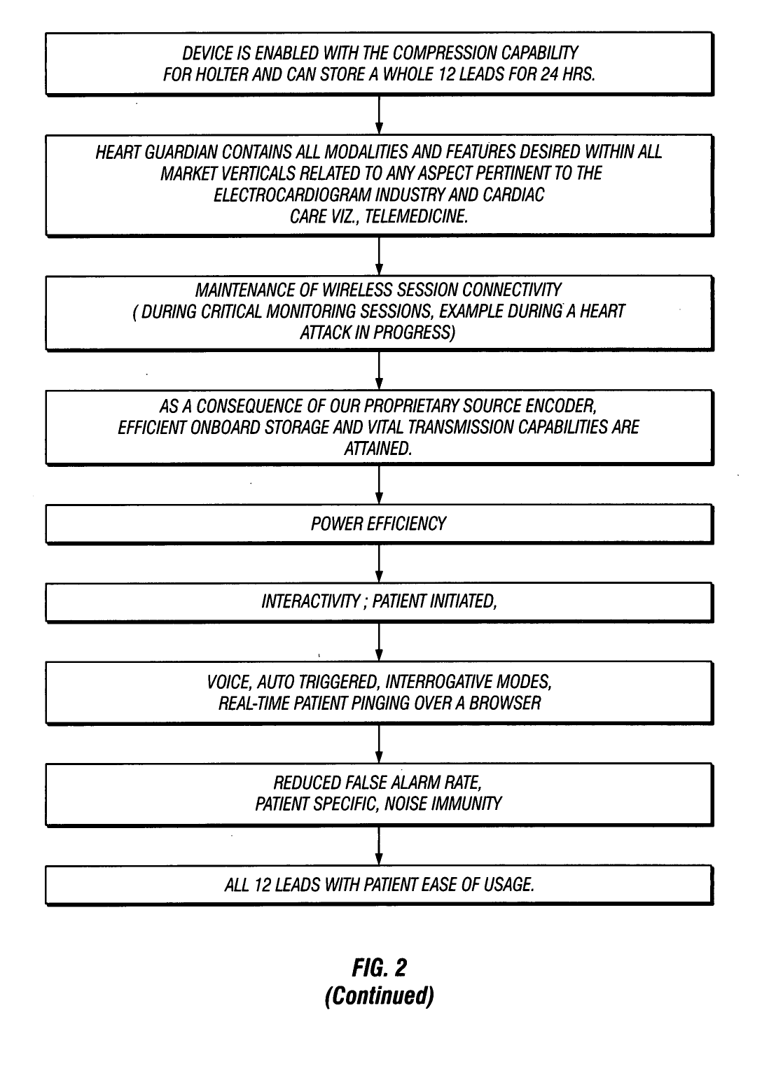

### SUMMARY OF THE INVENTION

The present invention provides a lightweight, portable, multi-functional device comprising highly efficient algorithms for detection and transmission.

A significant feature of the invention is an algorithm for conditioning waveforms prior to being transmitted. This is effective in transforming infinite space-time domain into finite space samples into an easily implemented and manipulated processing domain.

It is a feature of the present invention to have individualized baseline monitoring algorithms that updates constituent parameters as time progresses minute by minute. This feature includes a self-dissimilarity time based arrhythmia detector with adjustable coefficients according to, time varying, pathological-physiological excursions utilizing both the Euclidian and Hamming norms.

It is a feature of the present invention that a device made according to the invention is capable of being adapted to utilize minimum resources, reduced complexity, power efficient arrhythmia detector algorithm for complex arrhythmia by extracting trends via a supervised maximum entropy processor. The device is simple and easy to use and operate requiring little instruction.

It is also a feature of the present invention that a device made according to the invention can take detected signals and compare them to baseline data that has been selectively retrieved and stored so that the detected signal can be compared against the baseline and any trend or anomaly may be transmitted. In one embodiment most thoroughly described below, this feature is more fully explained.

### DETAILED DESCRIPTION OF THE PREFERRED EMBODIMENT

The device of the present invention is designed to take individualized and supervised “Directed” and Blind based monitoring.

A real time power efficient algorithm for cardiac event monitoring which builds upon pathological-physiological processes will now be explained. The algorithm exploits the priori knowledge of individual patient data and tracks in real time evolutions via waveform excursion. The principal approach that underlies this algorithm is the Euclidean and Hamming metrics from the transformed space of the acquired waveform. Infinitely countable waveform space is reduced to manageable and easily manipulated finite discrete space. Real time excursions continually update the decision space. Higher standards of cardiac care are therefore feasible and can be maintained with interactivity that is enhanced and enabled by robust platform provided for the remote supervisory cardiac center.

A-Detection Rule: Decision Directed (Supervised)

1) Detect the complex or the primary as we discussed. First, what is the decision rule? Initial Decision rule is based on most distinguishing features of what is deemed, by physician or attending Nurse to be a complex or a primary data piece belonging to baseline set. Defining a discriminant of a complex is based on the revealing large features, which is the most cognitively intuitive characteristic of the complex. The Complex is parsed according to its excursion complexity. More segments are clearly associated with more excursions of one primary. The large features referred to earlier are modeled by their rate of change of individual segments and can be represented conveniently by the gradient. Individual gradients associated with segments from one complex define locally a finite set of consecutive ternary trend symbols. Trend symbols of a segment form a complex define a local metric referred to as the Hamming distance which jointly with the rest of the segments shall define a cumulative distance associated with that complex out of a total of M possible primaries. A decision or a detection rule associated with this complex is then designed from the set of these distances. One possible decision rule, for example, of a normally conducted pulse may utilize the QRS intrisicoid up and down sloping lines and hence only two segments may suffice. The nurse or the electrocardiographer will play an instrumental role during the training, or baselining, session.

Initial Scan (Baseline): 1-5 Minutes (Arbitrary)

0) AC noise and base line artifacts are first filtered out from data, so that a clean recording is taken. Noise segments may be discarded and hence precluded from compromising statistics obtained by algorithm over all data in the diary.

1) The attendant nurse/technician visually scans the patient's pre-existing diary for events while preparing patient and assessing cardiac rhythm. First and foremost, selected candidates of complexes of dominant rhythm are selected, then secondary and anomalous events are detected by their individual excursions and classified as “points of trends”.

2) The candidate complex is enlarged for closer examination, and to better determine and select points of trends of candidate complexes. For each complex, the attendant nurse/technician begins from left to right and perform clicks corresponding to pertinent excursions of each complex that will be used to determine the both the Euclidean and the Hamming distance.

Within each segment there may be some variability, from one complex to another of the same sort, as to the number of trend symbols within this segment hence affecting locally the segment Hamming distance which in turn will ultimately vary the cumulative Hamming distance of that complex.

Due to variability, the nurse can test the goodness of this initial representation viz., Hamming distance, by visually inspecting the goodness of the detection algorithm over, the next few complexes of the same class within a screen.

The algorithm consists of 2 phases:

First Phase:

1. The Training Phase: Here the nurse visually identifies the primaries.

2. The primaries are a set of complexes such that SKI=SK1 . . . SKM, where, KI=1I . . . MI; K=1: M and IεZk<=∞(+ve set of integers). SKI is defined as the ensemble of all primary rhythms within a record.

3. The nurse also identifies significant points of the primary such that the program can now determine the trend of the primary and the number of excursions for each primary. The number of excursions is 1 greater than the number of significant points that the nurse selects on each complex of a primary. The point suggest that from the 1 st point the program needs to scan all samples prior to the 1st sample until a change in trend is observed. Similarly, the last point suggests that the program needs to scan all points after it until a change in the trend is detected. All the defining points from all the complexes of a primary that the nurse had clicked on, are then stored. The excursions on each primary are defined by Ni: iεZk<=∞(+ve set of integers);

4. Using the stored points for each primary and their locations (the index number associated with them), the program calculates the diff1KIˆ, diff2KIˆ, . . . diffNKIˆ, the hammingwindowKIˆ and σiKI for diff1KIˆ, diff2KIˆ, . . . diffNKIˆ and hammingwindowKIˆ i.e. (σiKI; i: 1 . . . N+1) (where, ˆ indicates average value).

5. Using diff1KIˆ, diff2KIˆ, . . . diffNKIˆ, the hammingwindowKIˆ and σiKI for diff1KIˆ, diff2KIˆ, . . . diffNKIˆ and hammingwindowKIˆ, the program scans the completer diary and identifies each of the KI complexes using the same algorithm as defined in steps 1, 2, 3 of the 2nd Phase: Detection and Alarm Phase, and stores them in memory.

6. Using these stored complexes the program recomputes diff1KIˆ, diff2KIˆ, . . . diffNKIˆ and hammingwindowKIˆ, along with the σiKI for diff1KIˆ, diff2KIˆ, . . . diffNKIˆ and hammingwindowKIˆ, i.e. (σiKI; i: 1 . . . N+1) so that the lim (μi, σi)=0;

i→∞

SKI

7. These are then stored into the DSP and the patient is sent home

2nd Phase: Detection and Alarm Phase;

1. The DSP takes in the input samples and starts forming M number of windows, where M=number of primaries.

2. It first checks for the Euclidean distance between the windowKI and its hammingwindowKIˆ. If the Euclidean distance is within the σN+1KI (variance for hammingwindowKIˆ), it goes to step 3. Else, it shifts the hamming window by 1 and then goes back to get the next set of samples.

3. It checks if the diff1KI, diff2KI, . . . diffNKI for the windowKI, is within the σ1KI . . . σNKI (variance for the diff1KIˆ, diff2KIˆ, . . . diffNKIˆ), and the number of excursions that satisfy this criteria should be greater than or equal to at least 50% of the total number of excursions for that primary (NKI). Mathematically, this can be represented as follows:
(diff1KIεdiff1KIˆ∩diff2KIεdiff2KIˆ∩ . . . diffNKIεdiffNKI)U(σ1KI . . . σNKI)>=0.5NKI
4. If so, it computes the energy between consecutive QRS and the heart rate.
The Heart Rate is computed using the formula:
HR=300/(((#of samples between 2 QRS)/sampling rate)*5).
5. If the first QRS is detected, then it starts storing all input samples until the next QRS is detected. Else, it shifts the hamming window by 1 and then goes back to get the next set of samples.
6. The Alarm phase is used to monitor the following:
Difference in energies between 2 QRS.
Heart Rate
If no QRS within 1 second.
   7. The Heart rate variation is because of minor variation to original primaries because of circadian cycle. This variation is reported back to the nurse, so that it can be decided whether the primaries information had changed.
   8. The energy variations are of two types:
Due to Isolated event, such as the PVC. In this case an emergency signal is sent back to the nurse.

If a new primary is detected: KI extended or reduced or same but replaced. In this case, an emergency signal is sent back to the nurse and she can update the primaries information.

Functions

| 1) | Intialize Variables( ) |
|---|---|
| | { |
| | Update_Alert_Para=0; //This value is set to 1 when the modem receives detection |

algorithm parameters from the server on the air for an update.

Channel_no=0; //Indicates the A/D channel number being acquired.
Value=0;
Total_channels; //Total A/D channels being.
Sample_count=0; //Count of the sample being stored in Flash.
for (int i=0; i<Total_channels; i++)

Prev_sample [channel] =0; //Array to store A/D samples for processing.
Sessions_Recorded=0; //Indicates the number of sessions recorded in Flash.
Alert=0; //This parameter is set to 1 when the detection algorithm detects

an alert.

Modem_On=0; // If the connection is established successfully then this parameter

is 1.

Flash_Write=0; // If this parameter is 1 then session has to be recorded in Flash.
Flash_Read=1; //Read from Flash
Loc =0; //This is a pointer which indicates the location in flash where a sample has

to be stored.

N=108000; // This is the Total Number of bytes corresponding to 15 min. When

the data has to be stored in Flash, these many bytes are stored to make one complete
session.

Total_Bytes_Transmitted=0;
//This indicates the total bytes transmitted by the modem. The ongoing session

can be terminated if the monitoring center sends an acknowledgement indicating a
successful session or when this parameter reaches a value=1080000 bytes corresponding
to 15 min worth of data being transmitted.

Ack_Session_Successful=0; //Acknowledgement received from the monitoring

center indicating a successful session. When the acknowledgement is received this
parameter is set to 1. This acknowledgment can be identified if a string
“Acknowledgement for a session” is received by the COM port.

Ack_Block_Successful=0; //Acknowledgement received from the monitoring

center for every block of data being transmitted. The block length can be determined.
When an acknowledgment for a block length is received this value is set to 1. This
acknowledgment can be identified if a string “Acknowledgement for a block” is received
by the COM port.

Block_Length=5*1200 bytes; //Indicates after how many bytes of transmission,

“Ack_Block_Successful” should be received from the monitoring center.

Landline_Modem=0; // If this value is 1 then Landline modem is selected for

communication else the wireless modem is used.

Wireless_Modem=1; // If this value is 1 then Wireless modem is selected for

communication. Default Wireless modem is used.

Push=0; //When once the connection has been established this value becomes

1.After that even if the user presses the push button it will have no effect until this value
becomes 0.This value is made 0 only when the connection is terminated.

Buffer_0_Write=0;
Buffer_1_Write=0;
In this program there are 2 buffers Buffer_0 and Buffer_1 defined at some known

memory location in RAM. When the flag “Buffer_0_Write” is 1 the A/D data is stored in
Buffer_0. When “Buffer_1_Write” flag is 1 the data is stored in Buffer_1. This is so that
when the modem is sending from One buffer and if A/D interrupt comes the acquired A/D
data will be stored in the other buffer. This is done so that we do not overwrite on the
same buffer if modem has not sent all the values from that buffer.

Buffer_0_Read=0;
Buffer_1_Read=0;
//Similarly, in this program there are 2 status flags Buffer_0_Read and

Buffer_1_Read. The DSP sends the data from Buffer_0 to the modem when
Buffer_0_Read is 1 else it sends from Buffer_1_Read.

Block_Cnt=0;
Wait_Ack=0; //When this value is 1 then the program waits for the string

“Ack_Block_Successful” from the monitoring center.

Start=0; // This is the location in Flash from which data has to be read and

transmitted.

Initialize_Channel=0;
Continue=0;
Sessions_Recorded=0;// This values indicates the total number of sessions

recorded in flash.

Sessions_Sent=0;// This values indicates the total number of sessions transmitted

out of the total recorded sessions in flash.

Sessions_Complete=0; // This values indicates the total number of sessions that's

have been transmitted including the sessions stored in flash and sessions which were
transmitted real time.

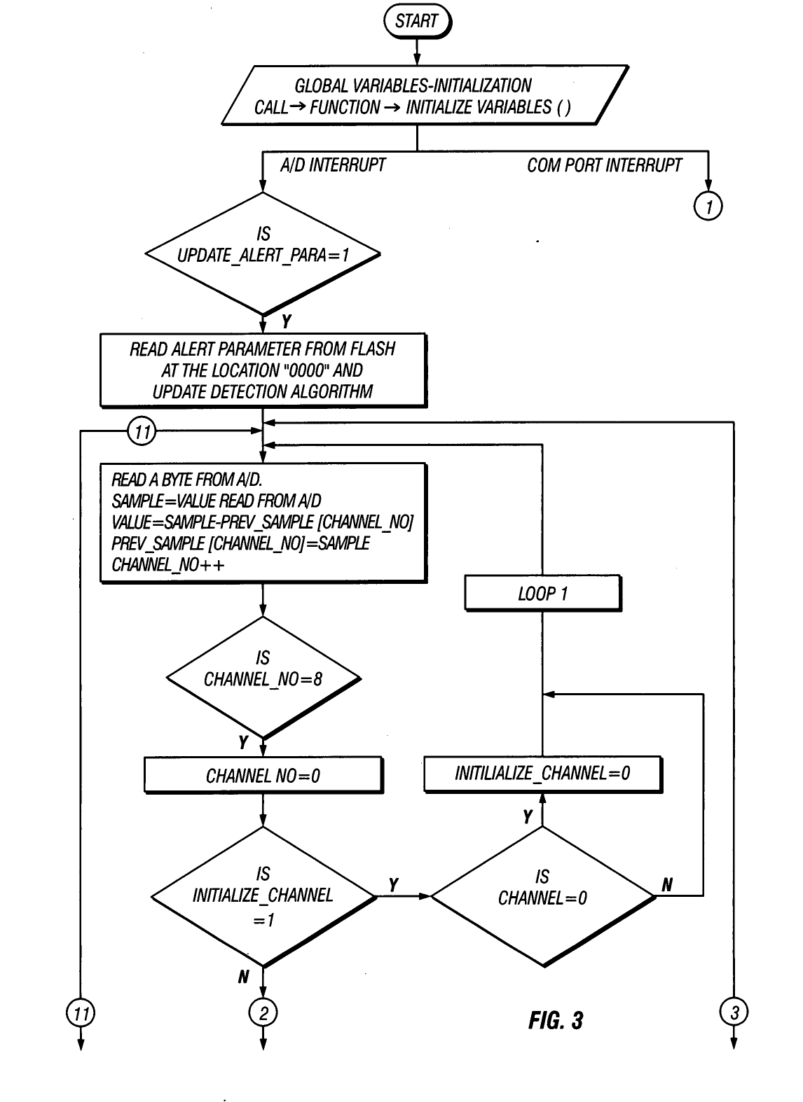

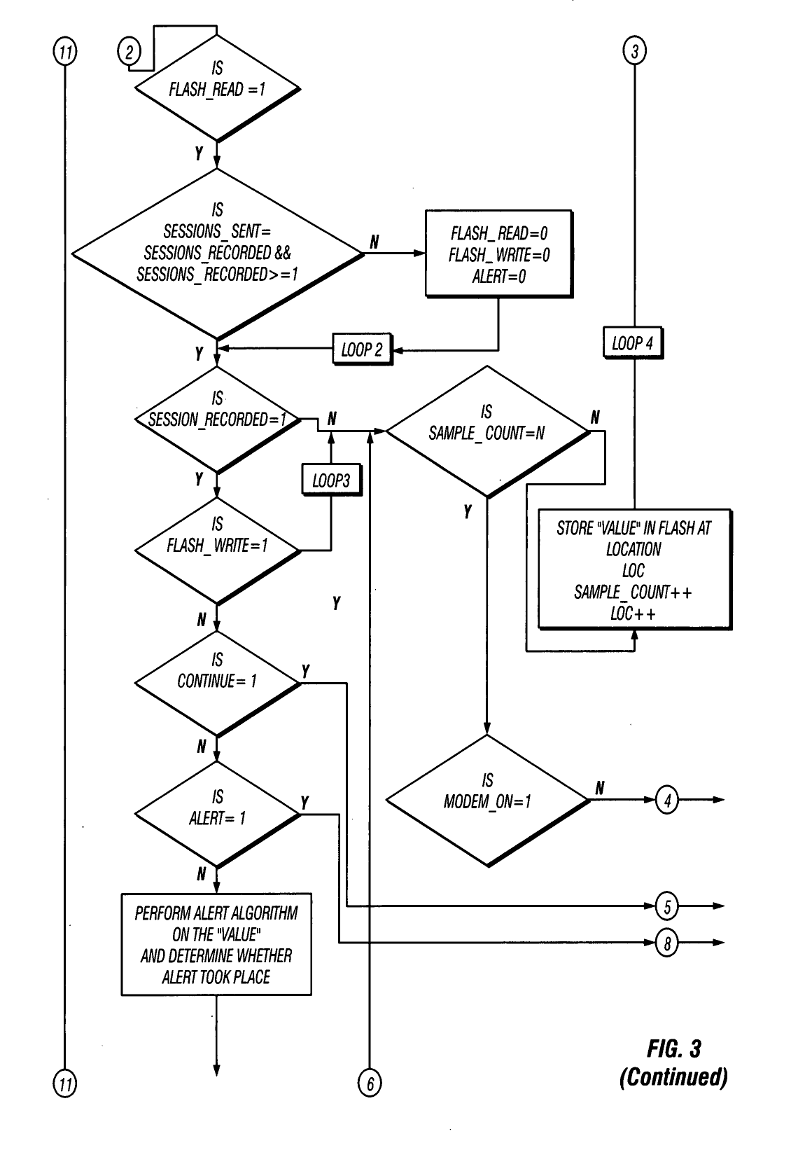

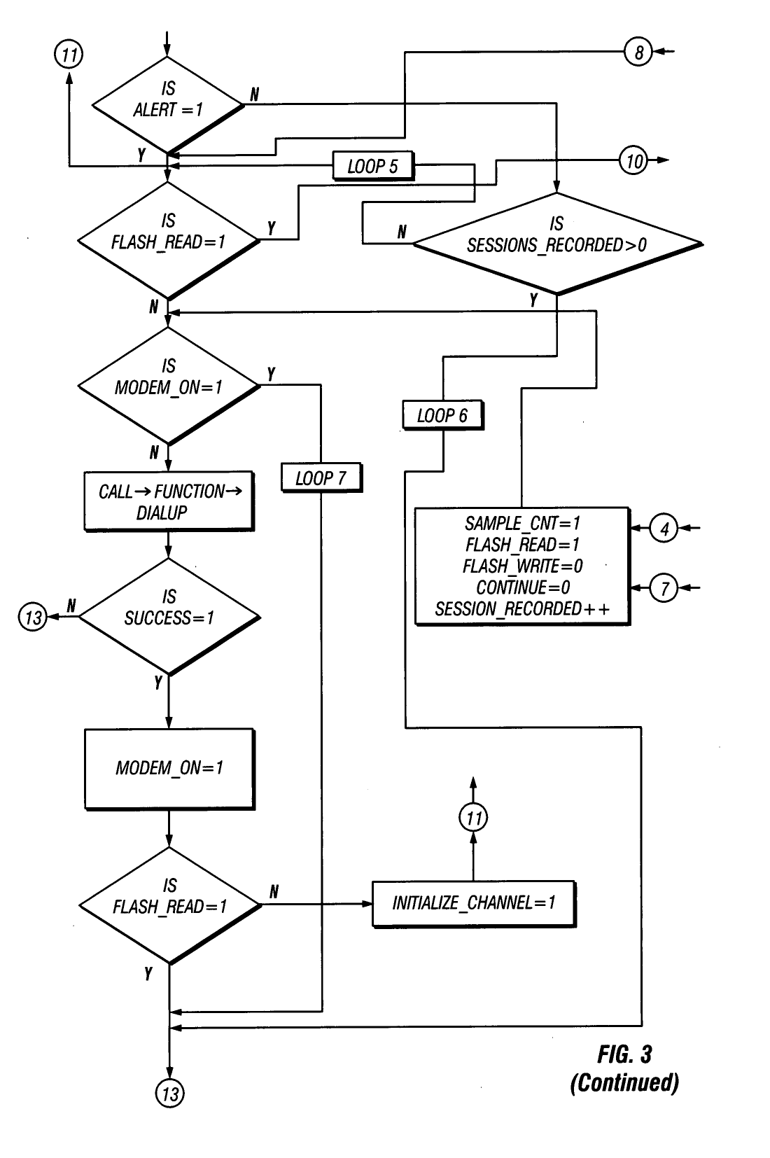

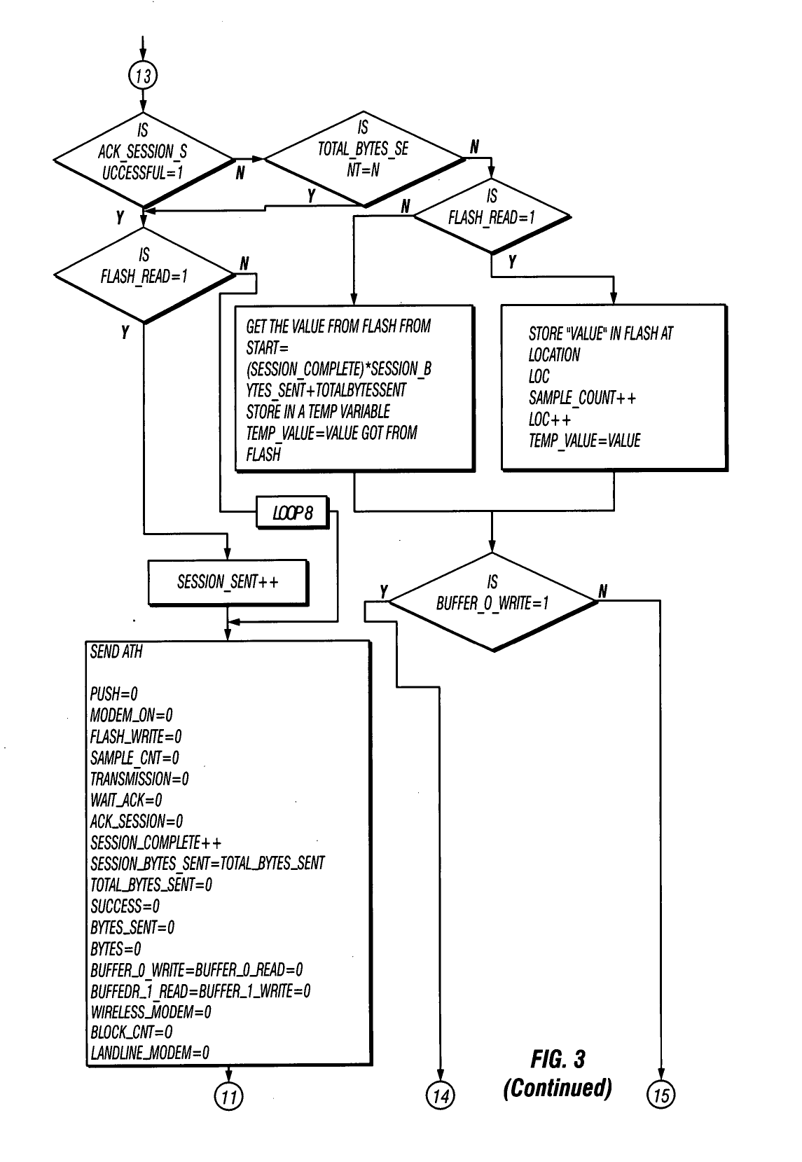

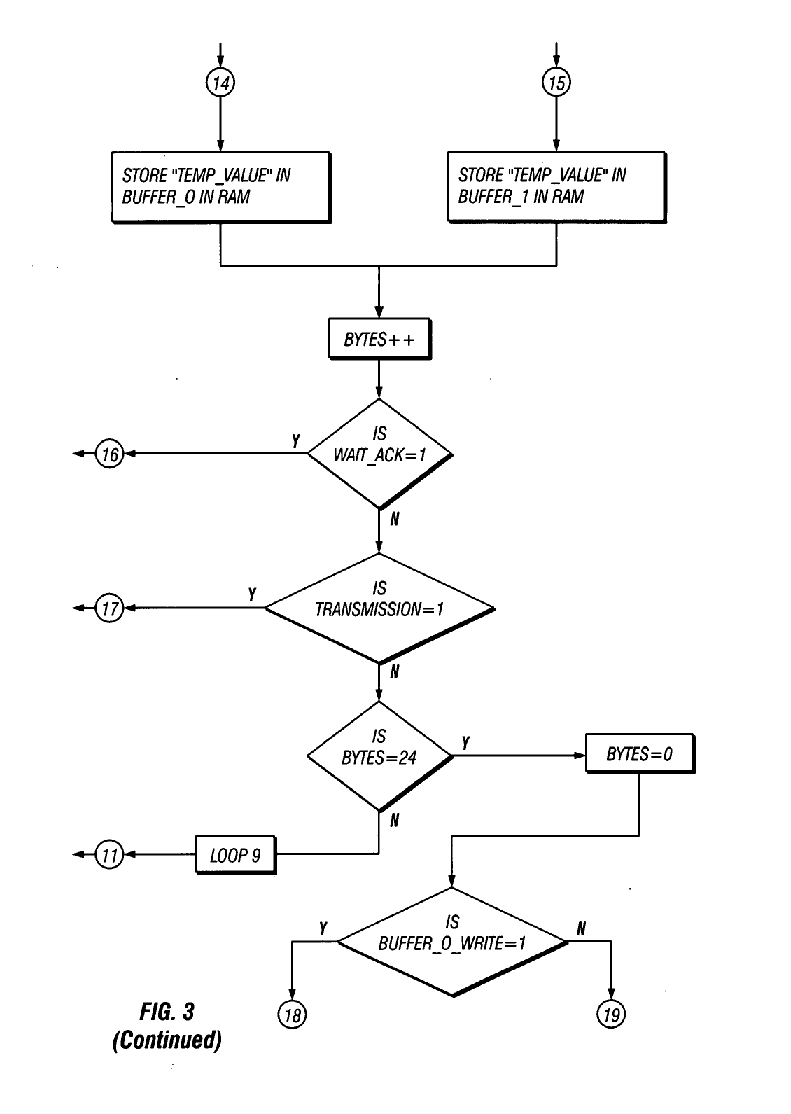

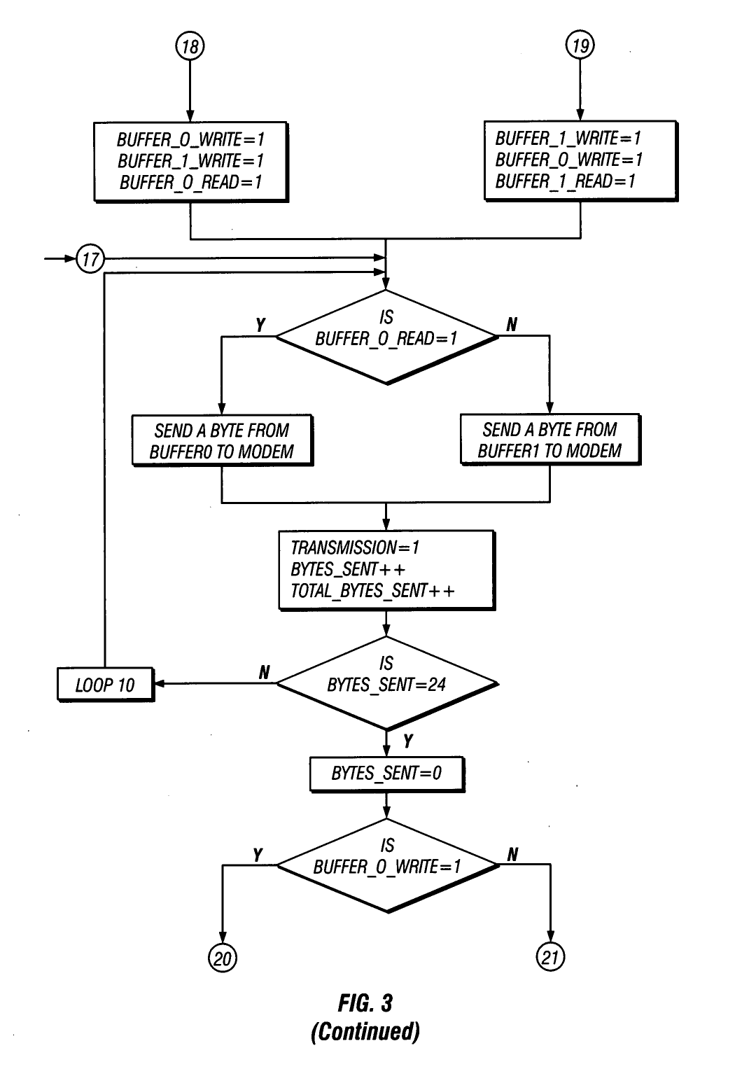

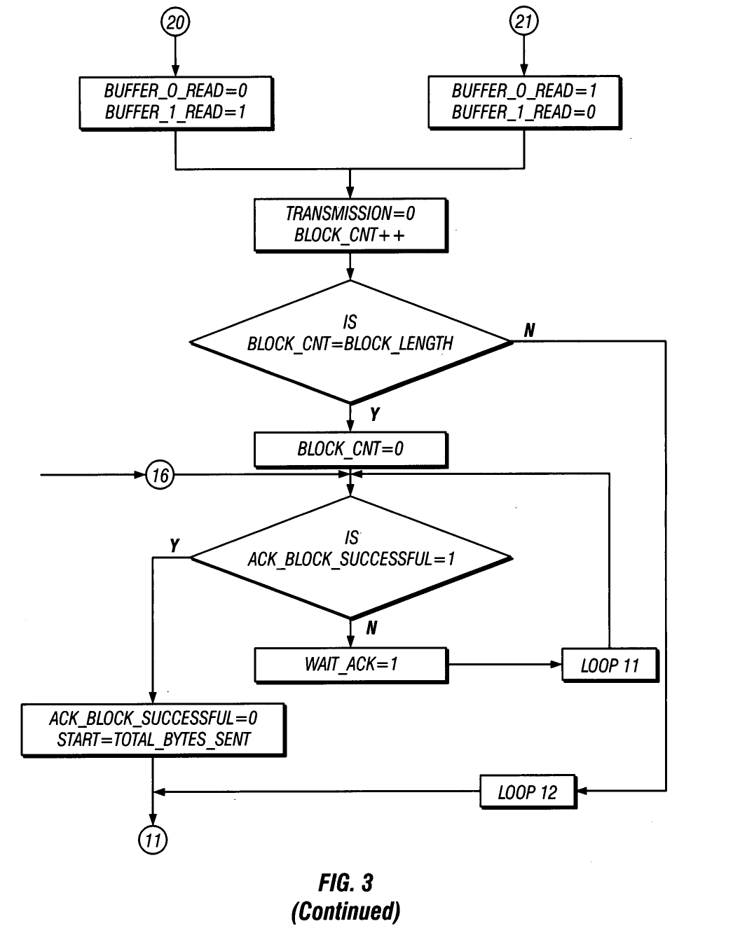

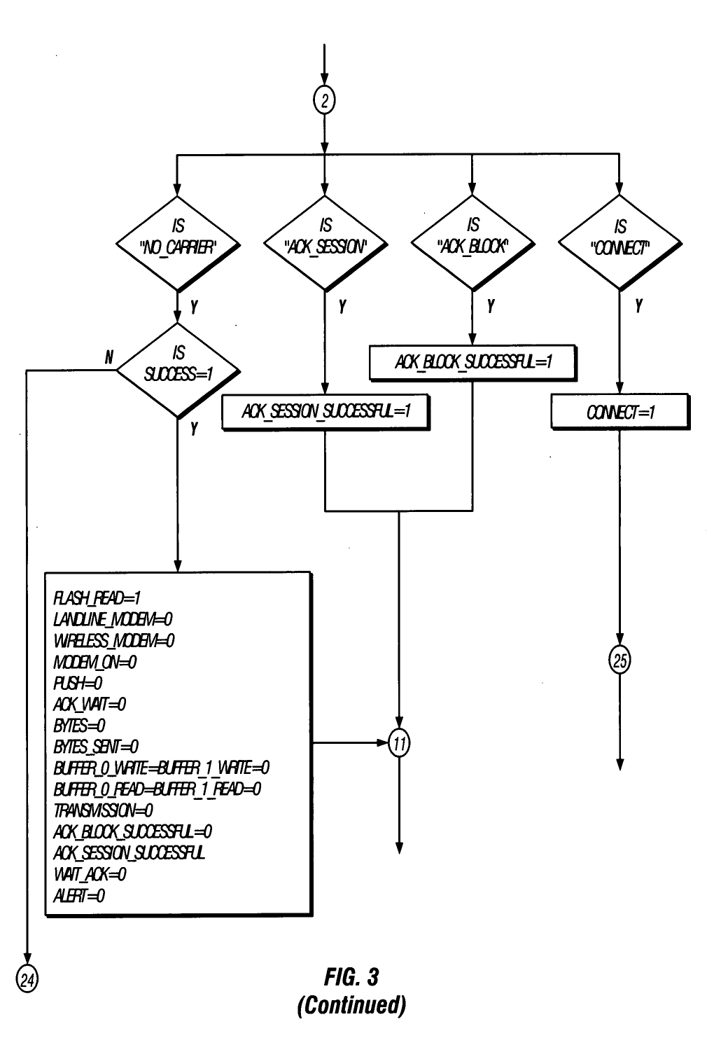

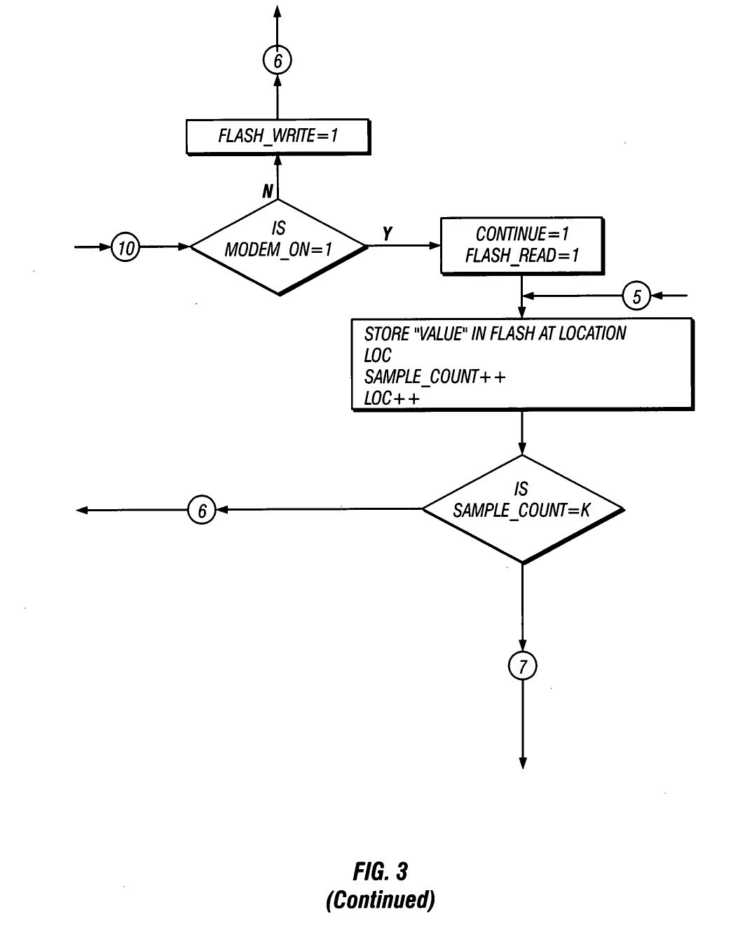

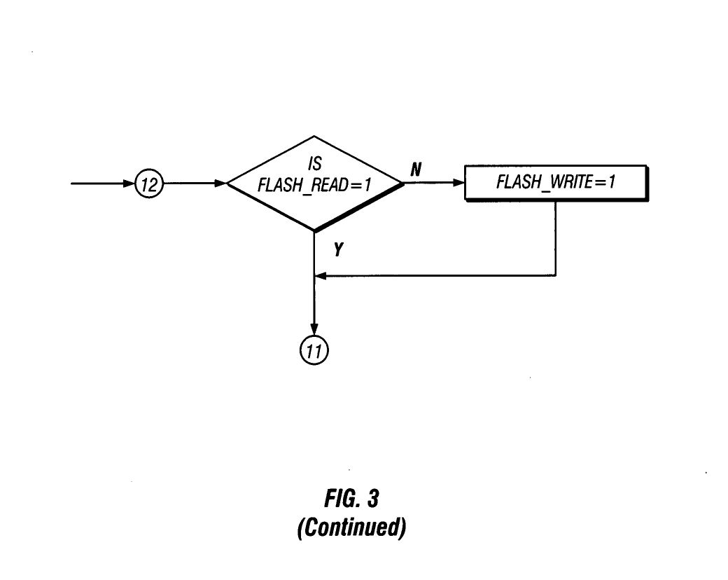

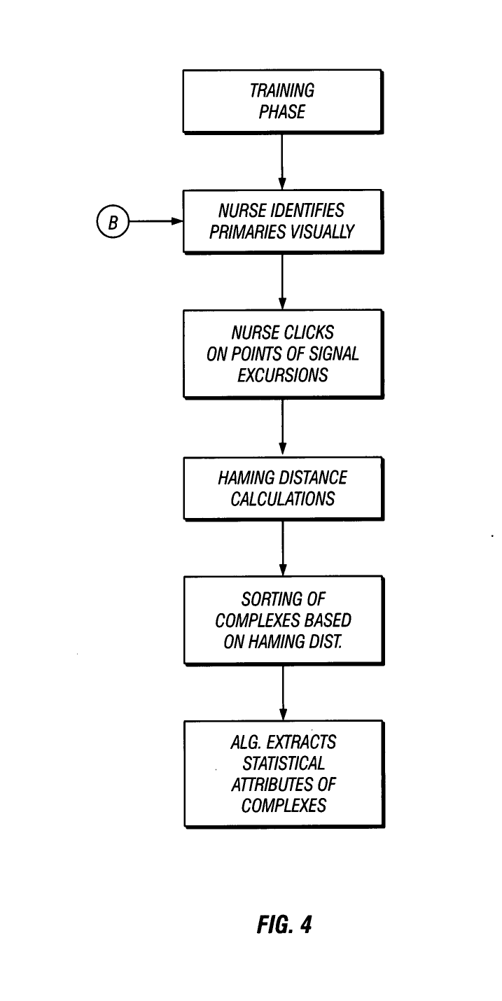

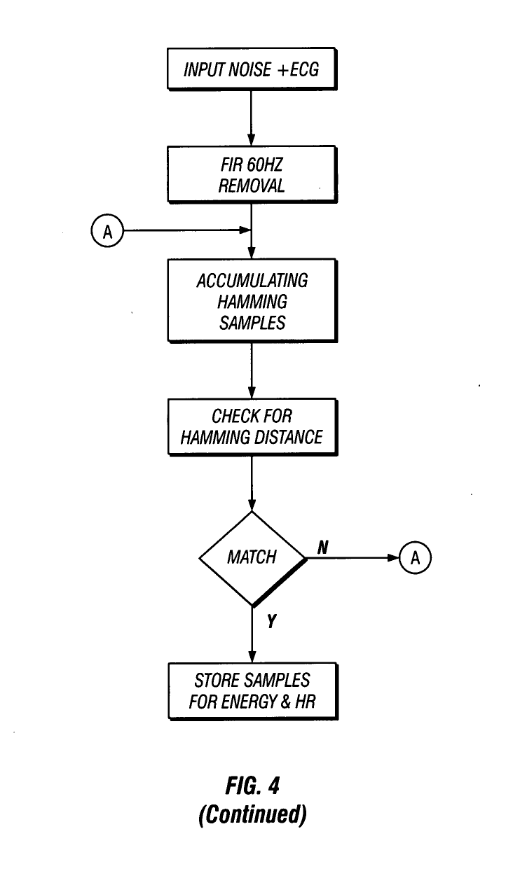

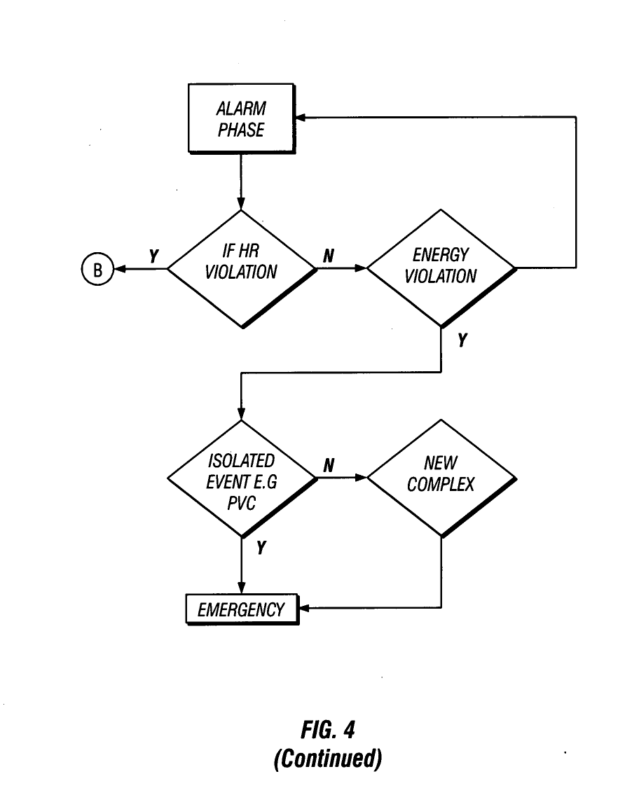

}

| | |
|---|---|
| 2) | Dialup Function is illustrated in FIG. 3. |

{
This function is called whenever we want to establish a call. There are 2 modems. One is
a landline modem and one a wireless modem. By default, the wireless modem is used
hence the variable “Wireless Modem” is 1. We initially check for the variable “Push”. If
this value is 0 then only the ATD function will be called. This is to avoid call of ATD
function due to accidental or intentional push of the button when a session has been
established. Thus when “Push” is 0 we look whether “Landline Modem” variable is 1.If
yes then we call ATD. If we receive a string “Connect” at the COM Port then we make
“Push 1”. Thus, now even if someone pushes the push button, the function ATD will
never be called until and unless the session breaks down or gets successfully completed.
If however we do not receive the String “Connect” at the COM port and rather receive the
String “No carrier”, we try establishing a call using the Wireless modem. We always
check for the signal strength when using the wireless modem. If the command AT+CSQ
gives value >5 we go ahead for the command ATD and the same procedure continues.
}

Connectors

Connector 1: This connector controls the Com Port Interrupts (See Document Comport.Doc”). Depending upon the type of Interrupt received on the Com port the variables are Set or Reset.

Connector 4: When a session is successfully recorded in flash then we see if “Modem_On”=1. If no then we increment the “Sessions_Recorded” by 1 and call the dial up function.

Connector 5: This connector is used when we are transmitting a session from flash and an event occurs. Thus we need to record this new event and at the same time continue transmitting the previous events.

Connector 10 and Connector 6: When we get an alert and “Flash_Read” is 1 which indicates that we need to store data in flash either because we lost a connection or because we could not establish one. Thus now we check if “Modem_On” is 1 (See Document 2). i.e. whether a connection is already established. If not it indicates that we need to store data in flash and then once N bytes are stored we need to transmit. Thus connector 6 loops backs to the place for storing data in flash.

Connector 11: This brings us back to the data acquiring and monitoring mode.

Connector 12: When we receive “No Carrier” at the COM port due to a failure in establishing a call, we need to store the event in flash. Thus we check if “Flash_Read” is 1. If yes we go to connector 11 else we make “Flash_Write=1” indicating now we need to store data in flash and the go to connector 11.

Connector 14: Indicates that we need to store the byte in Buffer_0 (See Functions.Doc for definition of Buffer_0).

Connector 15: Indicates that we need to store the byte in Buffer_0 (See Functions.Doc for definition of Buffer_1).

Loops

Loop 1: Whenever the Data is read from A/D and an alert is observed we need to start storing the data. In order to do this we need to make sure that we start storing data from channel 1 of A/D. This is because the alert may have resulted from a byte which corresponds to channel 5. However, we intend to start storing data from channel 1. So we will skip bytes from channel 5-8 and then come back to channel 1 and start the process of storing data.

Loop 2: When we store data in flash we increment a variable called Sessions_Recorded. Also when we successfully transmit a session from flash a variable called Sessions_Sent gets incremented. Thus when these two variables become equal we make Flash_Read=0 and Flash_Write=0. This indicates that no more sessions are there in flash and so we need not read from flash.

Loop 3: This loop indicates that whenever “Flash_Write” is lwe need to store data in flash completely before we transmit.

Loop 4: This loop ensures that the number of samples stored in flash is N (See Document Functions for these variables)

Loop 5: This loop is used when we do not detect any alert. We check to see if there are any stored sessions (Sessions_Recorded>1) in flash that need to be transmitted. If not we again go in the monitoring mode.

Loop 6: This loop is used when we do not detect any alert. We check to see if there are any stored sessions (Sessions_Recorded>1) in flash that need to be transmitted. If yes we need to transmit them.

Loop 7: In this loop we see if “Modem_On”=1. This variable will be 1 if a connection is established. Thus if a connection already exists then we need not call Dialup Function

Loop 8: If we are transmitting data real-time that is not from flash then “Flash_Read” will be 0. In this case we need no increment “sessions_sent” as it is incremented only when we transmit data from flash.

Loop 9: We always store data in blocks. In this invention, the current convention of collecting 24 bytes in RAM and then giving it to the modem is followed. Thus this loop ensures that we have 24 bytes in Buffer_1 and Buffer_0 alternatively before we give it to the modem. One skilled in this art will understand that there can be many variations on this theme.

Loop 10: Whenever a byte from Buffer_0 or Buffer_1 is given to modem and if an A/D interrupt comes, the interrupt is served and the control comes back from connector 17 to here to ensure that we transmit all the 24 bytes collected in the buffer (either Buffer_0 or Buffer_1 to the modem).

Loop 11: When we transmit x bytes to the modem corresponding to “Block_Length”, we need to wait for an acknowledgement from the monitoring center. Thus connector 16 brings us in this loop.

Loop 12: When we transmit x bytes to the modem corresponding to “Block_Length”, we need to wait for an acknowledgement from the monitoring center. This loop checks if we have transmitted bytes equal to “Block_Length” to the modem. Since we have not collected bytes equal vent to “Block_Length” to the modem, control goes back to connector 11.

As described above, and within this design spirit of efficient algorithms for the detection of anomalous event of rhythmic generators, the underlying impetus behind this disclosure lies within redefining the classical understanding of statistical or probabilistic stationarity of waveforms especially bio-physiological signals. Several prior art devices employ detection algorithms based on stochastic processors but labored to achieve some form of classical process stationarity in their attempt so as to force a previously well understood characterization to suit biophysiological signals such as ECG and EEG. In most if not all of these processors, little or no regards was afforded to the wealth of prior knowledge of the ECG, EEG waveforms and they were treated and hence processed as any given random waveform. Examples are terms like weak stationarity and pseudo stationarity and cyclo-stationarity. While these characterizations are well understood from digital signaling and when describing natural phenomenon, they fail short in characterizing pathological and physiological waveforms adequately.

The forgoing is a generalization to a more intuitive understanding of the individualized form of stationarities. Stationarity here is used to refer to long term record with randomness, possible random experiment outcomes, to HRV, and the occasional appearance of intermittent PVC or other events deemed benign, not warranting urgent action, and hence “within base line or new baseline”. Other terms such as short term stationarity are also used in characterizing time varying cellular channels

Because the prior knowledge plays a central role in the in the detection algorithms and the simplicity in transmission, the desired form of stationarity is, what I refer to as, a generalized pseudo stationarity that relies on several inherent and interactive factors.

Modifications of the apparatus, procedures and conditions disclosed herein that will still embody the concept of the improvements described should readily suggest themselves to those skilled in the art, and are intended to be encompassed within the spirit of the invention presently disclosed herein as well as the scope of the appended claims.

What is claimed is:

1. Ambulatory patient monitoring apparatus comprising:
a portable housing comprising:
at least one physiological data input device operative to gather physiological data of said patient;
location determination circuitry operative to determine geographic location information of said patient;
cellular telephone communications circuitry for communicating said physiological data and said geographic location information to a central health monitoring station;
voice communications circuitry whereby said patient conducts voice communications with a clinician at said central health monitoring station;
digital signal processing circuitry for processing signals associated with any of said physiological data input device, said location determination circuitry, said cellular telephone communications circuitry, and said voice communications circuitry; and
control circuitry for controlling any of said digital signal processing circuitry, said physiological data input device, said location determination circuitry, said cellular telephone communications circuitry, and said voice communications circuitry.

2. Apparatus according to claim 1 wherein said at least one physiological data input device is assembled within said housing.

3. Apparatus according to claim 1 wherein said at least one physiological data input device is at least partially external to said housing.

4. Apparatus according to claim 3 wherein said external portion of said at least one physiological data input device is connected to said via housing via a connector.

5. Apparatus according to claim 1 wherein said location determination circuitry comprises GPS circuitry.

6. Apparatus according to claim 1 wherein said control circuitry operates said physiological data input device continuously.

7. Apparatus according to claim 1 wherein said control circuitry operates said physiological data input device upon initiation by said patient.

8. Apparatus according to claim 1 wherein said memory comprises preset parameters adapted for comparison with said physiological data.

9. Apparatus according to claim 8 wherein said control circuitry is operative to determine whether said physiological data are within preset parameters.

10. Apparatus according to claim 9 wherein said control circuitry is operative to initiative contact with said central health monitoring station when said physiological data are determined to be outside of said preset parameters.

11. Apparatus according to claim 1 wherein said memory comprises preprogrammed instructions for output to said patient via either of a display and a speaker.

12. A system for monitoring a patient, the system comprising:
a central health monitoring station; and
a portable housing for use by said patient, the portable housing comprising:
at least one physiological data input device operative to gather physiological data of said patient;
location determination circuitry operative to determine geographic location information of said patient;
cellular telephone communications circuitry for communicating said physiological data and said geographic location information to said central health monitoring station;
voice communications circuitry whereby said patient conducts voice communications with a clinician at said central health monitoring station;
digital signal processing circuitry for processing signals associated with any of said physiological data input device, said location determination circuitry, said cellular telephone communications circuitry, and said voice communications circuitry; and
control circuit for controlling any of said digital signal processing circuitry, said physiological data input device, said location determination circuitry, said cellular telephone communications circuitry, and said voice communications circuitry wherein said control circuitry comprises a memory for storing any of said physiological data.

13. A method for monitoring a patient, the method comprising:
providing a portable housing for use by said patient, the portable housing comprising:
at least one physiological data input device operative to gather physiological data of said patient;
location determination circuitry operative to determine geographic location information of said patient;
cellular telephone communications circuitry for communicating said physiological data and said geographic location information to said central health monitoring station;
voice communications circuitry whereby said patient conducts voice communications with a clinician at said central health monitoring station;
digital signal processing circuitry for processing signals associated with any of said physiological data input device, said location determination circuitry, said cellular telephone communications circuitry, and said voice communications circuitry; and
control circuitry for controlling any of said digital signal processing circuitry, said physiological data input device, said location determination circuitry, said cellular telephone communications circuitry, and said voice communications; and
gathering physiological data of said patient; and
storing said physiological data in a memory wherein storing said physiological data comprises simultaneously storing a first portion of said physiological data in said memory in FIFO fashion and a second portion of said physiological data in said memory that is write-protected with respect to said first portion;
determining the geographic location of said patient; and
communicating said physiological data and said geographic location to said central health monitoring station.

14. A method according to claim 13 and further comprising:
analyzing said physiological data; and
providing a response based on said physiological data.

15. A method according to claim 13 wherein said gathering step is performed in response to activation by said patient.

16. A method according to claim 13 and further comprising activating an alarm prior to said activation by said patient.

17. A method according to claim 13 wherein said gathering step is performed independently from activation by said patient.

18. A method according to claim 13 wherein said communicating step is performed in response to activation by said patient.

19. A method according to claim 13 wherein communicating said physiological data and said geographic location is performed independently from activation by said patient upon said memory becoming full.

20. A method according to claim 19 and further comprising clearing a portion of said memory corresponding to said physiological data that has been communicated to said central health monitoring station.

21. A method according to claim 13 wherein said communicating step comprises: determining whether said physiological data are outside of preset parameters; and establishing a communications link with said central health monitoring station when said physiological data are determined to be outside of said preset parameters.

22. A method according to claim 14 wherein said providing a response step comprises voice-communication an instruction to said patient.
